(19) United States
(12) Patent Application Publication
(10) Pub. No.: US 2010/0264042 A1
(43) Pub. Date: Oct. 21, 2010

(54) METHOD OF MONITORING GAS COMPOSITION

(75) Inventors: Mark Varney, Hampshire (GB); Michael Garrett, Surrey (GB)

Correspondence Address: Kimberly A. Chasteen, PO Box 1243, Yorktown, VA, 23692, US

(73) Assignee: ANAXSYS TECHNOLOGY LTD, Nr Working, Surrey (GB)

(21) Appl. No.: 12/740,774

(22) PCT Filed: Oct. 24, 2008
(86) PCT No.: PCT/GB2008/003628

§ 371 Date: Jun. 16, 2010

(30) Foreign Application Priority Data
Oct. 30, 2007 (GB) 0721179.0

(51) Int. Cl. G01N 33/497; G01N 27/26

(52) U.S. Cl. 205/783; 205/785.5

(57) ABSTRACT

A method of determining the general or a specific condition of a subject is disclosed, the method comprising measuring the concentration of each of a plurality of components in a single sample of the gas stream exhaled by the subject; and generating information regarding the concentration of each of the plurality of components such that the concentrations of the components are directly comparable. The method is particularly suitable for assessing the condition of the respiratory system of a subject. The method preferably employs electrochemical sensors to measure the concentration of the target components in the exhaled gas stream.

The present invention relates to a method for detecting the changes in concentration of at least two gases in a gas stream, in particular with respect to time, from a single sample. The present invention is particularly directed to a method of the aforementioned kind for analysing the composition of a sample of a gas stream exhaled by a subject, so as to obtain and indication of the condition of the lungs of the subject.

The analysis of the gas streams exhaled by subjects is known in the art. Such analysis is carried out, for example, with a view to identifying the onset or existence of certain respiratory conditions, for example asthma, chronic obstructive pulmonary disease (COPD) and the like. To this end, methods and devices are known for measuring the concentration of key components in the exhaled gas stream, in particular water vapour or carbon dioxide. In particular, it has been proposed to measure the concentration of carbon dioxide or water vapour in the exhaled gas stream of a subject to produce a capnogram or humidogram, from which the condition of the respiratory system of the subject may be assessed.

Surprisingly, it has now been found that the measurement of the concentration of both water vapour and carbon dioxide in a single sample of a gas stream exhaled by a subject can provide very useful information indicating the overall condition of the lungs and airways of the subject.

According to a first aspect of the present invention, there is provided a method of determining the general or specific condition of a subject, the method comprising:

measuring the concentration of each of a plurality of components in a single sample of the gas stream exhaled by the subject; and

generating information regarding the concentration of each of the plurality of components such that the concentrations of the components is directly comparable.

The method of the present invention may be used to analyse and assess both the general condition of a subject and specific conditions that the subject may be afflicted with. In particular, the method is particularly useful in assessing general and specific conditions relating to the respiratory system of the subject, including the lungs and respiratory tract of the subject. Other conditions that may be assessed include those affecting the cardiovascular system and endocrine system of the subject.

In general, when investigating phenomena such as restricted transport of gases, changes in concentration sometimes exhibit chromatographic behaviour of the gaseous components. It has now been found that the behaviour of gases exhaled by a human or animal subject vary from component to component in the exhaled gas stream. The method of this invention monitors the concentration of the components of a changing gas stream from a single sample line, relating component concentrations to time.

Gas diffusion rates vary in known fashion and are related to the conditions, in particular temperature and pressure, and are inversely proportional to the density of the gas in question. Consequently if a mixture of gases meets a restriction the lower density gases will permeate faster than those of higher density. Further if the concentrations in the mixture are varying upstream of the restriction then the rates of change measured downstream of the restriction can yield useful information about both this and the restriction. The respiratory system contains many restrictions in the flow path for gases passing into and out of the lungs and, as a result, the aforementioned effects are present in the gas streams inhaled and exhaled by a subject. In particular, the composition of a gas stream leaving an inner part of the lungs of the subject will be different to the composition of the gas stream as exhaled through the mouth or nose of the subject, due to these diffusional effects.

The present invention relates to methods of measuring these differences and rates of change and recording them for purposes of comparison with each other to detect changes in the restriction or rate of concentration change in incident gas, or both. To achieve this it is important that the measurements of the concentrations of the components of the gas stream are made in a manner that allows the values for the concentrations to be correlated with one another, to take into account differences in the flowpaths leading to the individual sensors, time delays in the measurements or the like, which may result in changes in the overall composition of the gas sample, due to the aforementioned diffusional effects. In particular, it is important that the gases are detected by sensors and by instruments which have a faster response rate than that of the change being measured. It is also important that sampling is coincident or that any time difference between the detectors is known, such that any concentration change attributable to this can be readily determined and taken into account when producing data relating to the relative concentrations of the components being measured.

A preferred method of sampling is to have the relevant detectors exposed to the sample stream side by side so that the readings are directly time comparable. Alternatively, the stream may be split immediately prior to the detectors and subsequently recombined. The detector and related instrumentation should be capable of processing and storing the incoming information and producing the results graphically or if necessary superimposed.

Some sampling will be associated with an intermittent gas stream and in this instance it may be advantageous to also record flowrate of the gas stream, if necessary time related to the detectors. Again this would be part of the graphical interface or may be mathematically processed to normalise the other data. The system can also be used to advantage in systems where the gas flow is intermittent and reversing, and also by utilising part of the reverse flow to recalibrate the sensors.

An application for the present invention is in the measurement and analysis of the function of the lungs of a subject. The concentration of each of two or more gases being exhaled by a subject can be monitored over time and the changes in the concentrations, as well as the relative changes one to the other, can be used to provide an indication of the ventilator processes taking place as the patient exhales. The measured changes in concentration can be used to determine the lung function and assist in diagnosing the condition of the lungs and certain conditions affecting the respiratory system of the subject. In this case, the two components of the exhaled gas stream to be measured are most conveniently water vapour and carbon dioxide.

As noted, the method of the present invention comprises measuring the concentration of two or more components in the single gas sample under conditions whereby the values of the concentration may be directly compared with one another. The method may include measuring the concentration of the plurality of components over a period of time, in particular during the exhalation of the gas stream by the subject, an measuring the changes in the concentrations of the components. This method may include determining the rate of change of the concentrations of the components. Further, the method may include determining the ratio of two or more components present in the sample, together with, if desired, the rate of change of the ratios.

The method may employ any suitable form of sensor for measuring the concentration of the target components in the gas stream. A preferred sensor is an electrochemical sensor for measuring the concentration of at least one, preferably all of the target components. Most preferably, a separate sensor is employed for each target component. A preferred electrochemical sensor comprises a sensing element disposed to be exposed to the gas stream, the sensing element comprising a working electrode; a counter electrode; and a layer of ion exchange material extending between the working electrode and the counter electrode; whereby contact of the ion exchange layer with the gas stream forms an electrical contact between the working and counter electrodes. This form of sensor is particularly preferred for detecting water vapour and/or carbon dioxide.

In the present specification, references to an ion exchange material are to a material having ion exchange properties, such that contact with the components of a gas stream results in a change in the conductivity of the layer between the electrodes. The ion exchange material acts as the support medium for electrical conduction to occur. In particular, in the presence of water in the ion exchange material, it allows a hydrated ionic layer to form between the electrodes. The layer of ion exchange material provides a medium that is highly controllable and hydrates uniformly to provide a suitable medium for conduction to occur.

Suitable ion exchange materials for use in the preferred sensor are those having a high proton conductivity, good chemical stability, and the ability to retain sufficient mechanical integrity. The ion exchange material should have a high affinity for the species present in the gas stream being analysed, in particular for the various components that are present in the exhaled breath of a subject or patient.

Suitable ion exchange materials are known in the art and are commercially available products.

Particularly preferred ion exchange material are the ionomers, a class of synthetic polymers with ionic properties. A particularly preferred group of ionomers are the sulphonated tetrafluoroethylene copolymers. An especially preferred ionomer from this class is Nafion®, available commercially from Du Pont. The sulphonated tetrafluroethylene copolymers have superior conductive properties due to their proton conducting capabilities. The sulphonated tetrafluroethylene copolymers can be manufactured with various cationic conductivities. They also exhibit excellent thermal and mechanical stability and are biocompatible, thus making them suitable materials for use in the controlled electrode coating.

Other suitable ion exchange materials include polyether ether ketones (PEEK), poly(arylene-ether-sulfones) (PSU), PVDF-graft styrenes, acid doped polybenimidazoles (PBI) and polyphosphazenes.

The ion exchange material may be present in the sensor in the dry state. This is the case when the sensor is used to analyse the exhaled breath of a human or animal, where water vapour in varying amounts is present. Alternatively, the ion exchange material may be present with water in a saturated or partially-saturated state, in which case a dry gas stream may be analysed. In such a case, the output of the sensor will change in response to a change in the conductance of the ion exchange material, due to the dissolution of ions in the water present. In general, in the method of the present invention, water vapour will be present in the gas stream exhaled by a subject, which is then analysed directly to determine the concentration of various of the components present in the gas stream.

The thickness of the ion exchange material will determine the response of the sensor to changes in the composition of the gas stream in contact with the ion exchange layer. To minimize internal resistance within the sensor, it is preferred to use an ultra thin ion exchange layer.

The ion exchange layer may comprise a single ion exchange material or a mixture of two or more such materials, depending upon the particular application of the sensor.

The ion exchange layer may consist of the ion exchange material in the case the material exhibits the required level of chemical and mechanical stability and integrity for the working life of the sensor. Alternatively, the ion exchange layer may comprise an inert support for the ion exchange material. Suitable supports include oxides, in particular metal oxides, including aluminium oxide, titanium oxide, zirconium oxides and mixtures thereof. Other suitable supports include oxides of silicon and the various natural and synthetic clays.

In one particularly preferred embodiment, the ion exchange layer comprises, in addition to the ion exchange material and inert filler, if present, a mesoporous material. In the present specification, references to a mesoporous material are to a material having pores in the range of from 1 to 75 nm, more particularly in the range of from 2 to 50 nm. The mesoporous material provides a medium that is highly controllable and hydrates uniformly to provide a suitable medium for conduction to occur.

Suitable mesoporous materials for use in the sensor of the present invention are known in the art and commercially available, and include Zeolites. Zeolites are a particularly preferred component for inclusion in the ion exchange layer in the sensor of the present invention. One preferred zeolite is Zeolite 13X. Alternative mesoporous materials for use are Zeolite 4A or Zeolite P. The ion exchange layer may contain one or a combination of zeolite materials.

The granularity and thickness of the mesoporous material will determine the response of the sensor to changes in the composition of the gas stream in contact with the ion exchange layer. To minimize internal resistance within the sensor, it is preferred to use an ultra thin layer containing mesoporous material.

The mesoporous material is preferably dispersed in the ion exchange layer, most preferably as a fine dispersion. The mesoporous material is preferably dispersed as particles having a particle size in the range of from 0.5 to 20 μm, more preferably from 1 to 10 μm. In one embodiment, the mesoporous material is applied to the electrodes as a suspension of particles in a suitable solvent, with the solvent being allowed to evaporate to leave a fine dispersion of particles over the electrodes. Ion exchange material is then applied over the mesoporous dispersion. The mesoporous material is preferably applied in a concentration of from 0.01 to 1.0 g, as a uniform suspension in 10 ml of solvent, into which the electrode assembly is dipped one or more times. More preferably, the mesoporous material is applied in a concentration of from 0.05 to 0.5 g per 10 ml of solvent, especially about 0.1 g per 10 ml of solvent. Suitable solvents for use in the application of the mesoporous material are known in the art and include alcohols, in particular methanol, ethanol and higher aliphatic alcohols. Other suitable techniques for applying the mesoporous material include dry aerosol deposition, spray pyrolysis, screen printing, in-situ crystal growth, hydrothermal growth, sputtering, and autoclaving.

It has been found that the sparse population of mesoporous particles within the (continuous) ion exchange film affords the highest discrimination towards the detection of target species in the gas stream, in particular water vapour. Examination under a scanning electron microscope (SEM) of a preferred arrangement reveals a density of mesoporous particles such that each particle is, on average, distanced several body diameters, in particular from 1 to 5 body diameters, more preferably from 1 to 3 body diameters, away from the nearest neighbour.

It has also been found that thick films of ion exchange material degrade the performance of the sensor, as do thick continuous coats of the mesoporous material. In other words, it is the combination of a thin ion exchange layer and sparse population of mesoporous particles that performs best.

The sensor is particularly suitable for the detection of carbon dioxide, in particular carbon dioxide present in the exhaled breath of a person or animal. The sensor is also particularly suitable for the detection of water vapour in a gas stream. In the case of an exhaled gas stream, the measurement of the water vapour concentration exhaled by the subject allows an accurate determination of the carbon dioxide content of the exhaled breath to be determined. This feature renders the sensor particularly advantageous in the analysis of gas streams exhaled by humans and animals. In addition, the sensor provides a fast and accurate response to changes in the composition of the gas stream being analysed.

The sensor may be used at ambient temperature conditions, without the need for any heating or cooling, while at the same time producing an accurate measurement of the target substance concentration in the gas being analysed.

The sensor preferably comprises a housing or other protective body to enclose and protect the electrodes. The sensor may comprise a passage or conduit to direct the stream of gas directly onto the electrodes. In a very simple arrangement, the sensor comprises a conduit or tube into which the two electrodes extend, so as to be contacted directly by the gaseous stream passing through the conduit or tube. When the sensor is intended for use in the direct analysis of the breath of a patient, the conduit may comprise a mouthpiece, into which the patient may exhale. Alternatively, the sensor may be formed to have the electrodes in an exposed position on or in the housing, for direct measurement of a bulk gas stream. The precise form of the housing, passage or conduit is not critical to the operation or performance of the sensor and may take any desired form. It is preferred that the body or housing of the sensor is prepared from a non-conductive material, such as a suitable plastic.

As noted above, in one embodiment, the sensor relies upon the presence of water vapour in the gaseous stream being analysed to hydrate the ion exchange layer. If insufficient water vapour is present in the gaseous stream, the sensor may be provided with a means for increasing the water vapour content of the gas stream. Such means may include a reservoir of water and a dispenser, such as a spray, nebuliser or aerosol.

The electrodes may have any suitable shape and configuration. Suitable forms of electrode include points, lines, rings and flat planar surfaces. The effectiveness of the sensor can depend upon the particular arrangement of the electrodes and may be enhanced in certain embodiments by having a very small path length between the adjacent electrodes. This may be achieved, for example, by having each of the working and counter electrodes comprise a plurality of electrode portions arranged in an alternating, interlocking pattern, that is in the form of an array of interdigitated electrode portions, in particular arranged in a concentric pattern.

The electrodes are preferably oriented as close as possible to each other, to within the resolution of the manufacturing technology. The working and counter electrode can be between 10 to 1000 microns in width, preferably from 50 to 500 microns. The gap between the working and counter electrodes can be between 20 and 1000 microns, more preferably from 50 to 500 microns. The optimum track-gap distances are found by routine experiment for the particular electrode material, geometry, configuration, and substrate under consideration. In a preferred embodiment the optimum working electrode track widths are from 50 to 250 microns, preferably about 100 microns, and the counter electrode track widths are from 50 to 750 microns, preferably about 500 microns. The gaps between the working and counter electrodes are preferably about 100 microns.

The counter electrode and working electrode may be of equal size. However, in one preferred embodiment, the surface area of the counter electrode is greater than that of the working electrode to avoid restriction of the current transfer. Preferably, the counter electrode has a surface area at least twice that of the working electrode. Higher ratios of the surface area of the counter electrode and working electrode, such as at least 3:1, preferably at least 5:1 and up to 10:1 may also be employed. The thickness of the electrodes is determined by the manufacturing technology, but has no direct influence on the electrochemistry. The magnitude of the resultant electrochemical signal is determined principally by exposed surface area, that is the surface area of the electrodes directly exposed to and in contact with the gaseous stream. Generally, an increase in the surface area of the electrodes will result in a higher signal, but may also result in increased susceptibility to noise and electrical interference. However, the signals from smaller electrodes may be more difficult to detect.

The electrodes may be supported on a substrate. Suitable materials for the support substrate are any inert, non-conducting material, for example ceramic, plastic, or glass. The substrate provides support for the electrodes and serves to keep them in their proper orientation. Accordingly, the substrate may be any suitable supporting medium. It is important that the substrate is non-conducting, that is electrically insulating or of a sufficiently high dielectric coefficient.

The electrodes may be disposed on the surface of the substrate, with the layer of ion exchange material extending over the electrodes and substrate surface. Alternatively, the ion exchange material may be applied directly to the substrate, with the electrodes being disposed on the surface of the ion exchange layer. This would have the advantage of providing mechanical strength and a thin layer of base giving greater control of path length.

The ion exchange material is conveniently applied to the surface of the substrate by evaporation from a suspension or solution in a suitable solvent. For example, in the case of sulphonated tetrafluoroethylene copolymers, a suitable solvent is methanol. The suspension or solution of the ion exchange material may also comprise the inert support or a precursor thereof, if one is to be present in the ion exchange layer.

To improve the electrical insulation of the electrodes, the portions of the electrodes that are not disposed to be in contact with the gaseous stream (that is the non-operational portions of the electrodes) may be coated with a dielectric material, patterned in such a way as to leave exposed the active portions of the electrodes.

While the sensor operates well with two electrodes, as hereinbefore described, arrangements with more than two electrodes, for example including a third or reference electrode, as is well known in the art. The use of a reference electrode provides for better potentiostatic control of the applied voltage, or the galvanostatic control of current, when the “iR drop” between the counter and working electrodes is substantial. Dual 2-electrode and 3-electrode cells may also be employed.

A further electrode, disposed between the counter and working electrodes, may also be employed. The temperature of the gas stream may be calculated by measuring the end-to-end resistance of the electrode. Such techniques are known in the art.

The electrodes may comprise any suitable metal or alloy of metals, with the proviso that the electrode does not react with the electrolyte or any of the substances present in the gas stream. Preference is given to metals in Group VIII of the Periodic Table of the Elements (as provided in the Handbook of Chemistry and Physics, 62^{nd} edition, 1981 to 1982, Chemical Rubber Company). Preferred Group VIII metals are rhenium, palladium and platinum. Other suitable metals include silver and gold. Preferably, each electrode is prepared from gold or platinum. Carbon or carbon-containing materials may also be used to form the electrodes.

The electrodes of the sensor of the present invention may be formed by printing the electrode material in the form of a thick film screen printing ink onto the substrate: The ink consists of four components, namely the functional component, a binder, a vehicle and one or more modifiers. In the case of the present invention, the functional component forms the conductive component of the electrode and comprises a powder of one or more of the aforementioned metals used to form the electrode.

The binder holds the ink to the substrate and merges with the substrate during high temperature firing. The vehicle acts as the carrier for the powders and comprises both volatile components, such as solvents and non-volatile components, such as polymers. These materials evaporate during the early stages of drying and firing respectively. The modifiers comprise small amounts of additives, which are active in controlling the behaviour of the inks before and after processing.

Screen printing requires the ink viscosity to be controlled within limits determined by rheological properties, such as the amount of vehicle components and powders in the ink, as well as aspects of the environment, such as ambient temperature.

The printing screen may be prepared by stretching stainless steel wire mesh cloth across the screen frame, while maintaining high tension. An emulsion is then spread over the entire mesh, filling all open areas of the mesh. A common practice is to add an excess of the emulsion to the mesh. The area to be screen printed is then patterned on the screen using the desired electrode design template.

The squeegee is used to spread the ink over the screen. The shearing action of the squeegee results in a reduction in the viscosity of the ink, allowing the ink to pass through the patterned areas onto the substrate. The screen peels away as the squeegee passes. The ink viscosity recovers to its original state and results in a well defined print. The screen mesh is critical when determining the desired thick film print thickness, and hence the thickness of the completed electrodes.

The mechanical limit to downward travel of the squeegee (downstop) should be set to allow the limit of print stroke to be 75-125 um below the substrate surface. This will allow a consistent print thickness to be achieved across the substrate whilst simultaneously protecting the screen mesh from distortion and possible plastic deformation due to excessive pressure.

To determine the print thickness the following equation can be used:

Tw=(Tm×Ao)+Te

Where Tw=Wet thickness (um);

Tm=mesh weave thickness (um);

Ao=% open area;

Te=Emulsion thickness (um).

After the printing process the sensor element needs to be leveled before firing. The leveling permits mesh marks to fill and some of the more volatile solvents to evaporate slowly at room temperature. If all of the solvent is not removed in this drying process, the remaining amount may cause problems in the firing process by polluting the atmosphere surrounding the sensor element. Most of the solvents used in thick film technology can be completely removed in an oven at 150° C. when held there for 10 minutes.

Firing is typically accomplished in a belt furnace. Firing temperatures vary according to the ink chemistry. Most commercially available systems fire at 850° C. peak for 10 minutes. Total furnace time is 30 to 45 minutes, including the time taken to heat the furnace and cool to room temperature. Purity of the firing atmosphere is critical to successful processing. The air should be clean of particulates, hydrocarbons, halogen-containing vapours and water vapour.

Alternative techniques for preparing the electrodes and applying them to the substrate, if present, include spin/sputter coating and visible/ultraviolet/laser photolithography. In order to avoid impurities being present in the electrodes, which may alter the electrochemical performance of the sensor, the electrodes may be prepared by electrochemical plating. In particular, each electrode may be comprised of a plurality of layers applied by different techniques, with the lower layers be prepared using one of the aforementioned techniques, such as printing, and the uppermost or outer layer or layers being applied by electrochemical plating using a pure electrode material, such as a pure metal.

In use, the sensor is able to operate over a wide range of temperatures. However, the need for water vapour to be present in the gaseous stream be analysed requires the sensor to be at a temperature above the freezing point of water and above the dew point. The sensor may be provided with a heating means in order to raise the temperature of the gas stream, if required.

The method of operation of the electrochemical sensor requires that an electric potential is applied across the electrodes. In one simple configuration, a voltage is applied to the counter electrode, while the working electrode is connected to earth (grounded). In its simplest form, the method applies a single, constant potential difference across the working and counter electrodes. Alternatively, the potential difference may be varied against time, for example being pulsed or swept between a series of potentials. In one embodiment, the electric potential is pulsed between a so-called ‘rest’ potential, at which no reaction occurs, and a reaction potential.

In operation, a linear potential scan, multiple voltage steps or one discrete potential pulse are applied to the working electrode, and the resultant Faradaic reduction current is monitored as a direct function of the dissolution of target molecules in the water bridging the electrodes.

The measured current in the sensor element is usually small. The current is converted to a voltage using a resistor, R. As a result of the small current flow, careful attention to electronic design and detail may be necessary. In particular, special “guarding” techniques may be employed. Ground loops need to be avoided in the system. This can be achieved using techniques known in the art.

The current that passes between the counter and working electrodes is converted to a voltage and recorded as a function of the carbon dioxide concentration in the gaseous stream. The sensor responds faster by pulsing the potential between two voltages, a technique known in the art as ‘Square Wave Voltammetry’. Measuring the response several times during a pulse may be used to assess the impedance of the sensor.

The shape of the transient response can be simply related to the electrical characteristics (impedance) of the sensor in terms of simple electronic resistance and capacitance elements. By careful analysis of the shape, the individual contributions of resistance and capacitance may be calculated. Such mathematical techniques are well known in the art. Capacitance is an unwanted noisy component resulting from electronic artifacts, such as charging, etc. The capacitive signal can be reduced by selection of the design and layout of the electrodes in the sensor. Increasing the surface area of the electrodes and increasing the distance between the electrodes are two major parameters that affect the resultant capacitance. The desired Faradaic signal resulting from the passage of current due to reaction between the electrodes may be optimized, by experiment. Measurement of the response at increasing periods within the pulse is one technique that can preferentially select between the capacitive and Faradaic components, for instance. Such practical techniques are well known in the art.

The potential difference applied to the electrodes of the sensor element may be alternately or be periodically pulsed between a rest potential and a reaction potential, as noted above. The voltage may be pulsed at a range of frequencies, typically from sub-Hertz frequencies, that is from 0.1 Hz, up to 10 kHz. A preferred pulse frequency is in the range of from 1 to 500 Hz. Alternatively, the potential waveform applied to the counter electrode may consist of a “swept” series of frequencies. A further alternative is a so-called “white noise” set of frequencies. The complex frequency response obtained from such a waveform will have to be deconvoluted after signal acquisition using techniques such as Fourier Transform analysis. Again, such techniques are known in the art.

One preferred voltage regime is 0V (“rest” potential), 250 mV (“reaction” potential), and 20 Hz pulse frequency.

It is an advantage of the preferred sensor that the electrochemical reaction potential is approximately +0.2 volts, which avoids many if not all of the possible competing reactions that would interfere with the measurements, such as the reduction of metal ions and the dissolution of oxygen.

Embodiments of the present invention will now be described, by way of example only, having reference to the accompanying drawings, in which:

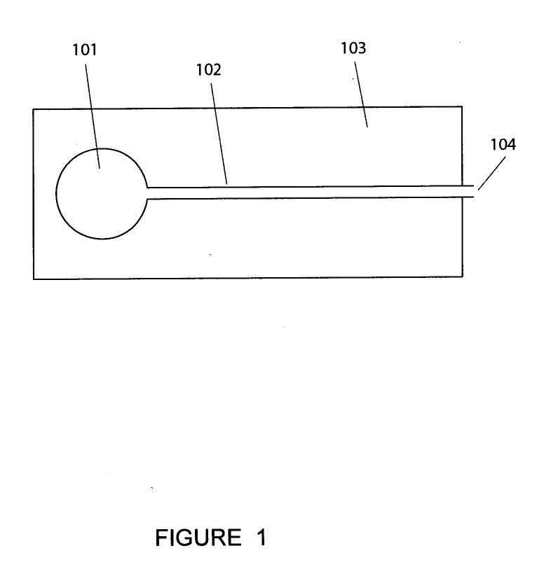

FIG. 1 is a schematic representation of the respiratory tract of a human or animal subject;

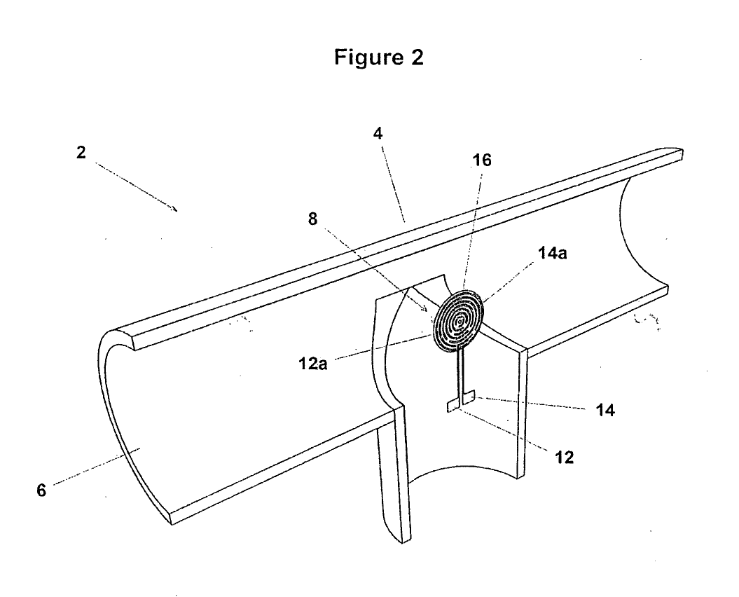

FIG. 2 is a cross-sectional representation of one embodiment of the preferred sensor for use in the method of the present invention;

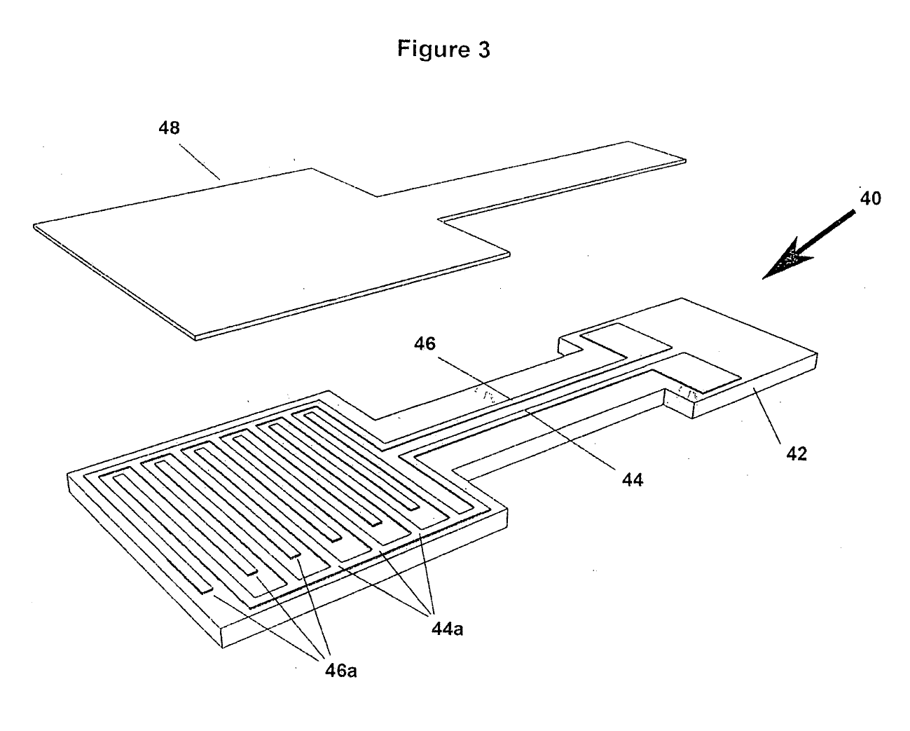

FIG. 3 is an isometric schematic view of a face of one embodiment of the preferred sensor element;

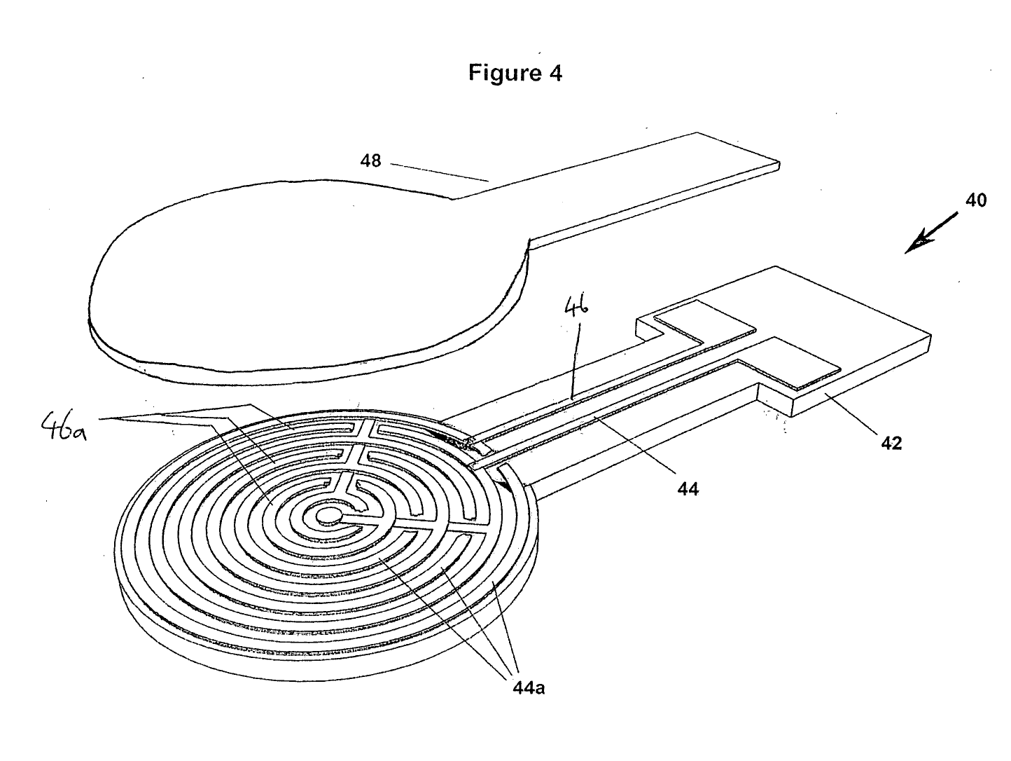

FIG. 4 is an isometric schematic view of an alternative embodiment of the preferred sensor element; and

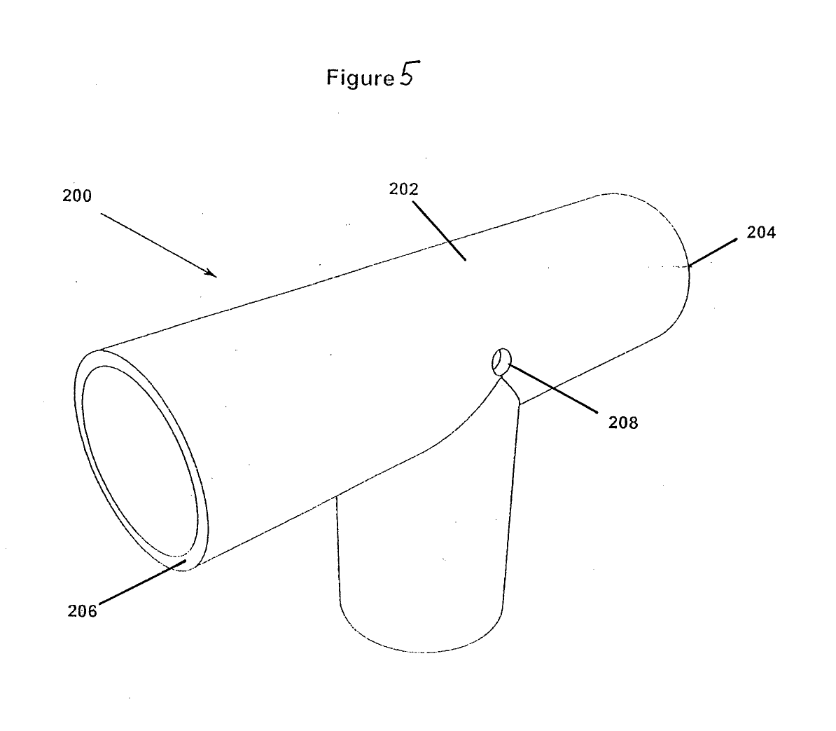

FIG. 5 is a schematic representation of a breathing tube adaptor for use in the sensor of the present invention.

Referring to FIG. 1, there is shown a schematic representation of the airway of a human subject showing an alveolus 101 connected to the atmosphere 104 via a long tube 102. The pressure difference across the wall of the alveolus 101 can be affected by the surrounding pressure within the chamber 103, which may be equal to, higher than or lower than the outside atmospheric pressure 104.

Referring to FIG. 2, there is shown a preferred electrochemical sensor. The sensor is for analyzing the carbon dioxide content and humidity of exhaled breath. The sensor, generally indicated as 2, comprises a conduit 4, through which a stream of exhaled breath may be passed. The conduit 4 comprises a mouthpiece 6, into which the patient may breathe.

A sensing element, generally indicated as 8, is located within the conduit 4, such that a stream of gas passing through the conduit from the mouthpiece 6 is caused to impinge upon the sensing element 8. The sensing element 8 comprises a support substrate 10 of an inert material, onto which is mounted a working electrode 12 and a reference electrode 14. The working electrode 12 and reference electrode 14 each comprise a plurality of electrode portions, 12a and 14a, arranged in concentric circles, so as to provide an interwoven pattern minimizing the distance between adjacent portions of the working electrode 12 and reference electrode 14. In this way, the current path between the two electrodes is kept to a minimum.

A layer 16 of insulating or dielectric material extends over a portion of both the working and counter electrodes 12 and 14, leaving the portions 12a and 14a of each electrode exposed to be in direct contact with a stream of gas passing through the conduit 4. The arrangement of the support, electrodes 12 and 14, and the solid electrolyte precursor is shown in more detail in FIGS. 3 and 4.

Referring to FIG. 3, there is shown an exploded view of a sensor element, generally indicated as 40, comprising a substrate layer 42. A working electrode 44 is mounted on the substrate layer 42 from which extend a series of elongated electrode portions 44a. Similarly, a reference electrode 46 is mounted on the substrate layer 42 from which extends a series of electrode portions 46a. As will be seen in FIG. 3, the working electrode portions 44a and the reference electrode portions 46a extend one between the other in an intimate, interdigitated array, providing a large surface area of exposed electrode with minimum separation between adjacent portions of the working and reference electrodes. A layer of ion exchange material 48 overlies the working and reference electrodes 44, 46.

The ion exchange material consists of Nafion®, a commercially available sulphonated tetrafluoroethylene copolymer.

The ion exchange material 48 is applied by the repeated immersion in a suspension or slurry of the Nafion® in a suitable solvent, in particular methanol. The pH will determine the ion exchanger characteristics of the Nafion®. It is possible to manufacture a Nafion® coating with principally H⁺, K⁺, Na⁺ and Ca²⁺ as the cationic exchanger. The sensor element is dried to evaporate the solvent after each immersion and before the subsequent immersion. Other materials may be incorporated into the ion exchange layer by subsequent immersion in additional solutions or suspensions. The number of immersions is determined by the required thickness of the ion exchange layer, and the chemical composition is determined by the number and variety of additional solutions that the sensor is dipped into.

It will be obvious that there are a number of other means whereby the thickness and composition of the coating may be similarly achieved, such as: pad, spray, screen and other mechanical methods of printing. Such techniques are well known in the field.

An alternative electrode arrangement is shown in FIG. 4, in which components common to the sensor element of FIG. 3 are identified with the same reference numerals. It will be noted that the working electrode portions 44a and the reference electrode portions 46a are arranged in an intimate circular array. The electrodes and substrate are coated in a layer of ion exchange material, as described above in relation to FIG. 3.

Turning to FIG. 5, an adaptor for monitoring the breath of a patient is shown. A sensor element is mounted within the adaptor and oriented directly into the air stream flowing through the adaptor, in a similar manner to that shown in FIG. 2 and described hereinbefore. The preferred embodiment illustrated in FIG. 5 comprises an adaptor, generally indicated as 200, having a cylindrical housing 202 having a male-shaped (push-fit) cone coupling 204 at one end and a female-shaped (push-fit) cone coupling 206 at the other. A side inlet 208 is provided in the form of an orifice in the cylindrical housing 202, allowing for the adaptor to be used in the monitoring of the tidal breathing of a patient. The side inlet 208 directs gas onto the sensor element during inhalation by a patient through the device. The monitoring of tidal breathing may be improved by the provision of a one-way valve on the outlet of the housing 202.

### EXAMPLE

The evaporation of water from the surface of the skin is significantly reduced by the presence of lipid molecular films. The rate of evaporation has been experimentally measured to be approximately 10.1×10−7 g·cm-2.s−1 (Shuzo Iwata, Michael Lemp, Frank Holly and Claes Dohlman “Evaporation rate of water from the precorneal tear film and cornea in the rabbit” Investigative Opthalmology December 1969, 613-619). The thickness of the lipid film is typically between 5 and 10 micron. This evaporation rate can be used to estimate the quantity of water passing across the surface of the lung wall, and exhaled through the mouth.

If the surface area of the lung conducting areas of the lung wall is assumed to be 200 cm², this gives an evaporation rate of approximately 2×10−4 g·s⁻¹. It is further assumed that a normal adult breathes at the rate of 0.5 l·s⁻¹. There is a moisture requirement of 30 g·m⁻³ to elevate the moisture content of inhaled breath at 20° C., resulting in 100% saturation within the exhaled breath at 37° C. It can thereore be calculated that normal tidal breathing therefore generates approximately 0.5×10−3 m3×30 g·m−3/1 s=15 mg water per second. It should be noted that this is an approximation, and that there are many other factors that influence the generation of water from the lung surface, and exhalation of water vapour.

If the natural occurrence of tidal breathing could be temporarily ignored, it can be expected this quantity of water would be passively transported along the length of the conducting airways towards the mouth (the ‘background’ evaporation rate of water). Furthermore, the rate of evaporation (diffusion) of carbon dioxide would be expected to be less, as it is a heavier molecule with a corresponding smaller diffusion coefficient.

1. A method of determining the general or a specific condition of a subject, the method comprising: measuring the concentration of each of a plurality of components in a single sample of the gas stream exhaled by the subject; and generating information regarding the concentration of each of the plurality of components such that the concentrations of the components are directly comparable.

2. The method according to claim 1, wherein the concentration of water vapour and carbon dioxide in the sample of the gas stream are measured.

3. The method according to claim 1, wherein the single sample of the gas stream is divided into a plurality of portions, each portion being used in the detection of one component in the gas stream.

4. The method according to claim 1, wherein the single sample of the gas stream is divided into a plurality of portions, each portion being used in the detection of one component in the gas stream and the portions are recombined after the concentration of the components has been measured.

5. The method according to claim 1, wherein the concentrations of the components are measured over a period of time during the exhalation of the sample and the changes in concentrations are measured.

6. The method according to claim 1, wherein the concentrations of the components are measured over a period of time during the exhalation of the sample and the changes in concentrations are measured and the rates of change of the concentrations with time are measured.

7. The method according to claim 1, wherein the ratio of the concentration of two or more components is determined.

8. The method according to claim 1, wherein the ratio of the concentration of two or more components is determined and the change in the ratio of the concentration of the components over a period of time during the exhalation of the sample is determined.

9. The method according to claim 8, wherein the rate of change of the ratio of concentration of the components is determined.

10. The method according to claim 1, wherein the concentration of one or more components is measured using an electrochemical sensor.

11. The method according to claim 1, wherein the concentration of each of the plurality of components is measured using an electrochemical sensor.

12. The method according to claim 1, wherein the concentration of one or more components is measured using an electrochemical sensor, the electrochemical sensor comprising:
a sensing element disposed to be exposed to the gas stream, the sensing element comprising:
a working electrode;
a counter electrode; and
a layer of ion exchange material extending between the working electrode and the counter electrode; whereby contact of the ion exchange layer with the gas stream forms an electrical contact between the working and counter electrodes.

13. The method according to claim 12, wherein the ion exchange material is an ionomer, especially a sulphonated tetrafluoroethylene copolymer.

14. The method according to claim 12, wherein the ion exchange layer comprises a mesoporous material.

15. The method according to claim 12, wherein the ion exchange layer is a material selected from the group consisting of a zeolite, in particular zeolite 13, zeolite 4A and a mixture thereof.

16. The method according to claim 12, wherein the ion exchange layer comprises a fine dispersion of mesoporous material.

17. The method according to claim 12, wherein the working electrode and counter electrode are in a form selected from the group consisting of a point, a line, rings and flat planar surfaces.

18. The method according to claim 12, wherein one or both of the working electrode and the counter electrode comprises a plurality of electrode portions.

19. The method according to claim 12, wherein one or both of the working electrode and the counter electrode comprises a plurality of electrode portions such that both the working electrode and the counter electrode comprise a plurality of electrode portions arranged in a manner selected from the group consisting of an interlocking pattern and a concentric pattern.

20. (canceled)

21. The method according to claim 12, wherein the surface area of the counter electrode is greater than the surface area of the working electrode.

22. (canceled)

23. (canceled)
